(19) United States
(12) Patent Application Publication
(10) Pub. No.: US 2004/0138438 A1
(43) Pub. Date: Jul. 15, 2004

(54) Cationised polysaccharide product

(76) Inventors: Fredrik Solhage, Boras (SE); Per-Ola Nilsson, Kristianstad (SE)

Correspondence Address: Michelle J. Burke, Akzo Nobel Inc. - Intellectual Property, 7 Livingstone Avenue, Dobbs Ferry, NY, 10522, US

(21) Appl. No.: 10/676,176

(22) Filed: Oct. 1, 2003

Related U.S. Application Data
(60) Provisional application No. 60/415,184, filed on Oct. 1, 2002.

(51) Int. Cl. C08B 37/00; A61K 7/06; A61K 7/11

(52) U.S. Cl. 536/18.7; 424/70.13

(57) ABSTRACT

The invention relates to a cationised polysaccharide product comprising a polysaccharide having at least one first substituent having an aromatic group and at least one second substituent having no aromatic group. The invention further relates to a cationised polysaccharide product comprising one or more polysaccharides having at least one first substituent having an aromatic group and one or more polysaccharides having at least one second substituent having no aromatic group. The present invention also relates to a method for the preparation of a cationised polysaccharide product comprising reacting one or more polysaccharides with at least one aromatic agent and at least one non-aromatic agent. The invention further relates to a method for the preparation of a cationised polysaccharide product comprising reacting a first polysaccharide with at least one aromatic agent, reacting a second polysaccharide with at least one second non-aromatic agent, and then mixing the polysaccharides obtained.

The present invention further relates to a process for production of paper from an aqueous suspension containing cellulosic fibres, and optionally fillers, which comprises adding to the suspension a cationised polysaccharide product comprising a polysaccharide having (i) at least one first substituent having an aromatic group, and (ii) at least one second substituent having no aromatic group, forming and draining the suspension on a wire. The invention also relates to a process for production of paper from an aqueous suspension containing cellulosic fibres, and optionally fillers, which comprises adding to the suspension a cationised polysaccharide product comprising (i) at least one polysaccharide having at least one first substituent having an aromatic group and (ii) at least one polysaccharide having at least one second substituent having no aromatic group, wherein one or both of the polysaccharides according to (i) and (ii) are cationic and/or amphoteric; forming and draining the suspension on a wire. The invention further relates to a process for production of paper from an aqueous suspension containing cellulosic fibres, and optionally fillers, which comprises separately adding to the suspension (i) at least one polysaccharide having at least one first substituent having an aromatic group; and (ii) at least one polysaccharide having at least one second substituent having no aromatic group, wherein one or both of the polysaccharides according to (i) and (ii) are cationic and/or amphoteric; forming and draining the suspension on a wire.

This application claims priority based on U.S. Provisional Patent Application No. 60/415,184, filed Oct. 1, 2002.

The present invention relates to a cationised polysaccharide product, which has one or more substituents having an aromatic group and one or more substituents having no aromatic group, a method for the preparation of the cationised polysaccharide product, use of the cationised polysaccharide product and a papermaking process in which the cationised polysaccharide product is used as an additives to an aqueous cellulosic suspension.

### BACKGROUND

In the papermaking art, an aqueous suspension containing cellulosic fibres, and optional fillers and additives, referred to as the stock, is fed into a headbox which ejects the stock onto a forming wire. Water is drained from the stock through the forming wire so that a wet web of paper is formed on the wire, and the web is further dewatered and dried in the drying section of the paper machine. Drainage and retention aids are widely used in the papermaking process and examples of such aids are cationic and amphoteric polysaccharides like cationic starches and cationic guar gums. The polysaccharides can be used alone or in combination with other polymers and/or with anionic microparticulate materials such as, for example, anionic inorganic particles like colloidal silica. Cationic and amphoteric polysaccharides are also widely used as dry strength agents.

International Patent Application WO 99/55964 discloses cationic or amphoteric polysaccharides having hydrophobic groups for use as additives in papermaking and as dry-strength agents for the paper produced.

U.S. Pat. Nos. 4,388,150, 4,755,259, 4,961,825, 5,127,994, 5,643,414, 5,447,604, 5,277,764, 5,607,552, 5,603,805, and 5,858,174, and European Patent No. 500,770 disclose the use of cationic and/or amphoteric polysaccharides and anionic inorganic particles as stock additives in papermaking.

The cationic groups of cationised polysaccharides can be obtained by the reaction of a polysaccharide with a quaternising agent. Examples of cationisation processes using such agents are known from U.S. Pat. Nos. 2,876,217, 3,422,087, 4,785,087, 5,827,372 and European Patent Nos. 303,039; 400,361; 737,210 and 874,000.

It would be advantageous to be able to provide drainage and retention aids with improved performance. It would also be advantageous to be able to provide a papermaking process with improved drainage and/or retention performance. It would further be advantageous to be able to produce a paper with improved dry strength properties.

### THE INVENTION

In accordance with the present invention there is generally provided a cationised polysaccharide product comprising at least one first substituent having an aromatic group and at least one second substituent having no aromatic group. There is also provided a cationised polysaccharide product comprising at least one first substituent having an aromatic group and at least one second substituent having no aromatic group, wherein the polysaccharide has a molar ratio of first substituent to second substituent within the range of from 10:1 to 1:10. There is also provided a cationised polysaccharide product comprising one or more polysaccharides having at least one first substituent having an aromatic group and one or more polysaccharides having at least one second substituent having no aromatic group. There is further provided a cationised polysaccharide product comprising at least one first substituent having an aromatic group and at least one second substituent having no aromatic group, wherein the polysaccharide has a degree of aromatic substitution (DS_{Ar}) within the range of from 0.0005 to 2.0 and a degree of non-aromatic substitution (DS_{non-Ar}) within the range of from 0.0005 to 2.0. There is also provided a cationised polysaccharide product obtainable by reacting one or more polysaccharides with at least one first aromatic agent and at least one second non-aromatic agent, wherein the first aromatic agent and second non-aromatic agent are reacted in a molar ratio within the range of from 10:1 to 1:10. There is also provided a cationised polysaccharide product obtainable by reacting a first polysaccharide with at least one first aromatic agent, reacting a second polysaccharide with at least one second non-aromatic agent, and then mixing the polysaccharides obtained.

The present invention also generally relates to a method for the preparation of a cationised polysaccharide product comprising reacting one or more polysaccharides with at least one aromatic agent and at least one non-aromatic agent. The invention further relates to a method for the preparation of a cationised polysaccharide product comprising reacting one or more polysaccharides with at least one first aromatic agent and at least one second non-aromatic agent, wherein the first aromatic agent and second non-aromatic agent are reacted in a molar ratio within the range of from 10:1 to 1:10. The invention further relates to a method for the preparation of a cationised polysaccharide product comprising reacting a first polysaccharide with at least one aromatic agent, reacting a second polysaccharide with at least one second non-aromatic agent, and then mixing the polysaccharides obtained.

The present invention further relates to a process for production of paper from an aqueous suspension containing cellulosic fibres, and optionally fillers, which comprises adding to the suspension a cationised polysaccharide product comprising a polysaccharide having (i) at least one first substituent having an aromatic group, and (ii) at least one second substituent having no aromatic group, forming and draining the suspension on a wire. The invention also relates to a process for production of paper from an aqueous suspension containing cellulosic fibres, and optionally fillers, which comprises adding to the suspension a cationised polysaccharide product comprising (i) at least one polysaccharide having at least one first substituent having an aromatic group and (ii) at least one polysaccharide having at least one second substituent having no aromatic group, wherein one or both of the polysaccharides according to (i) and (ii) are cationic and/or amphoteric; forming and draining the suspension on a wire. The invention further relates to a process for production of paper from an aqueous suspension containing cellulosic fibres, and optionally fillers, which comprises separately adding to the suspension (i) at least one polysaccharide having at least one first substituent having an aromatic group; and (ii) at least one polysaccharide having at least one second substituent having no aromatic group, wherein one or both of the polysaccharides according to (i) and (ii) are cationic and/or amphoteric; forming and draining the suspension on a wire.

### DETAILED DESCRIPTION OF THE INVENTION

The cationised polysaccharide product according to this invention has unexpectedly been found to improve the dry strength properties of paper produced. It has also been found that the cationised polysaccharide product according to the invention improves drainage and/or retention when used as additives to cellulosic suspensions in papermaking processes.

The cationised polysaccharide product according to this invention is suitably water-dispersible or, preferably, water-soluble. The cationised polysaccharide product can comprise one or more polysaccharides of the same or different type. The polysaccharides can be derived from any of the polysaccharides known in the art including, for example, starches, gums, celluloses, chitins, chitosans, glycans, galactans, glucans, pectins, mannans, dextrins, preferably starches and gums, and mixtures thereof. Examples of suitable starches include potato, corn, wheat, tapioca, rice, waxy maize, etc., preferably potato and corn. Examples of suitable gums are guar gums, tamarind gums, locust bean gums, tara gums, karaya, okra, acacia, xanthan gums etc., preferably guar gums.

The cationised polysaccharide product comprises one or more polysaccharides which are cationic and/or amphoteric, i.e. polysaccharides having one or more cationic groups. Examples of suitable cationic groups include sulphonium groups, phosphonium groups, tertiary amino groups and quaternary ammonium groups, preferably, quaternary ammonium groups. The polysaccharides may also contain one or more anionic groups. Examples of suitable anionic groups include phosphate, phosphonate, sulphate, sulphonate and carboxylic acid groups, preferably phosphate groups and sulphonate groups. The polysaccharides may also contain one or more non-ionic groups. If present, the anionic groups can be native or introduced by means of chemical treatment in conventional manner. Native potato starch contains a substantial amount of covalently bound phosphate monoester groups. In amphoteric polysaccharides, cationic groups are preferably present in a predominant amount.

The cationised polysaccharide product of this invention contains one or more polysaccharides having one or more substituents. As used herein, the term “substituent” means a group of atoms that is not present in the native polysaccharide but usually has been introduced by chemical treatment. Preferably the substituents are derived from an agent, as described herein, the substituent being formed by reacting the polysaccharide with the agent. As used herein, the term “first substituent” means a substituent which has an aromatic group, and the term “second substituent” means a non-aromatic substituent which has no aromatic group. The substituents can be attached to a heteroatom, e.g. oxygen or nitrogen, present in the polysaccharide. Heteroatoms such as oxygen or nitrogen can also be present in the substituents. In a preferred embodiment, the first substituent contains a heteroatom, preferably a nitrogen atom. In another preferred embodiment, the second substituent contains a heteroatom, preferably a nitrogen atom. The heteroatom of the first and second substituents can be charged, for example when it is nitrogen, e.g. ammonium ion, or potentially charged, e.g. nitrogen that is present in an amine group that can be rendered cationic by protonation; or uncharged, e.g. heteroatoms present in amide, ester or ether groups. The heteroatoms of the substituents can be attached to the polysaccharide for example via a chain of atoms. In a preferred embodiment of the invention, the cationised polysaccharide product comprises a polysaccharide which has two or more substituents, at least one first substituent and at least one second substituent.

In the first substituent having an aromatic group, the aromatic group can be selected from aryl and aralkyl groups, e.g. phenyl, phenylene, naphthyl, phenylene, xylylene, benzyl and phenylethyl; nitrogen-containing aromatic (aryl) groups, e.g. pyridinium and quinolinium, as well as derivatives of these groups where one or more substituents attached to said aromatic groups can be selected from hydroxyl, halides, e.g. chloride, nitro, and hydrocarbon groups having from 1 to 4 carbon atoms.

In a preferred embodiment of the invention, the first substituent having an aromatic group has the following general structural formula (I):

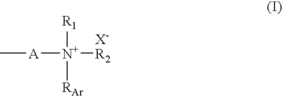

wherein A is a group attaching N to a polysaccharide, suitably a chain of atoms comprising C and H atoms, and optionally 0 and/or N atoms, usually an alkylene group with from 2 to 18 and suitably 2 to 8 carbon atoms, optionally interrupted or substituted by one or more heteroatoms, e.g. O or N, e.g. an alkyleneoxy group or hydroxy propylene group (—CH₂—CH(OH)—CH₂—); R₁ and R₂ are individually H or, preferably, a hydrocarbon group, suitably alkyl having from 1 to 3 carbon atoms, preferably 1 or 2 carbon atoms; R_{Ar} is an aromatic group containing at least 1 to 18 carbon atoms, suitably 1 to 15 and preferably 1 to 12 carbon atoms, preferably aralkyl groups, e.g. benzyl and phenylethyl groups; or, alternatively, R₁, R₂, and R_{Ar} together with N form a cyclic aromatic group, suitably having 5 to 12 carbon atoms; and X⁻ is an anionic counterion, usually a halide like chloride. Preferably, the first substituent having an aromatic group is —CH₂—CH(OH)—CH₂—N⁺((CH₃)₂)CH₂C₆H₅Cl⁻.

In the second substituent having no aromatic group, the substituent can be selected from aliphatic groups and alicyclic groups. Examples of suitable aliphatic groups and alicyclic groups include linear, branched and cyclic alkyl groups like methyl, ethyl; propyl, e.g. n-propyl and iso-propyl; butyl, e.g. n-butyl, iso-butyl and t-butyl; pentyl, e.g. n-pentyl, neo-penyl and iso-pentyl; hexyl, e.g. n-hexyl and cyclohexyl; octyl, e.g. n-octyl; decyl, e.g. n-decyl; dodecyl, e.g. n-dodecyl; tetradecyl and octadecyl.

In a preferred embodiment of the invention, the second substituent having no aromatic group has the following general structural formula (II):

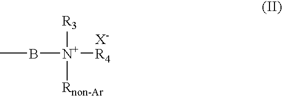

wherein B is a group attaching N to a polysaccharide, suitably a chain of atoms comprising C and H atoms, and optionally O and/or N atoms, usually an alkylene group with from 2 to 18 and suitably 2 to 8 carbon atoms, optionally interrupted or substituted by one or more heteroatoms, e.g. O or N, e.g. an alkyleneoxy group or hydroxy propylene group (—CH₂—CH(OH)—CH₂—); R₃ and R₄ are individually H or, preferably, a hydrocarbon group, suitably alkyl having from 1 to 3 carbon atoms, preferably 1 or 2 carbon atoms; R_{non-Ar} is a non-aromatic group containing at least 1 to 18 carbon atoms, suitably 1 to 15, preferably 1 to 12 and most preferably 1 to 4 carbon atoms, the group suitably being as defined above; or, alternatively, R₃ and R₄, optionally together with R_{non-Ar}, together with N form a cyclic group, suitably having 5 to 12 carbon atoms; and X is an anionic counterion, usually a halide like chloride. Preferably, the second substituent having no aromatic group is —CH₂—CH(OH)—CH₂—N⁺((CH₃)₃)Cl⁻.

Particularly suitable polysaccharide products according to the invention include polysaccharides with at least one first substituent having aromatic groups represented by the general structural formula (III):

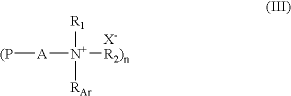

and at least one second substituent with no aromatic groups represented by the general structural formula (IV):

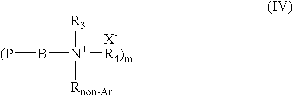

wherein P is a residue of a polysaccharide; A, B, R₁, R₂, R₃, R₄, R_{Ar}, R_{non-Ar} and X are as defined above, n and m are individually integers from about 1 to about 1,200,000, suitably from 5 to 600,000 and preferably from 6 to 300,000.

In a preferred embodiment of the invention, the cationised polysaccharide product comprises a polysaccharide having at least one cationic first substituent and at least one cationic second substituent, e.g. as described above. In another preferred embodiment, the cationised polysaccharide product comprises a first polysaccharide having at least one cationic first substituent, e.g. as described above, and a second polysaccharide having at least one cationic second substituent, e.g. as described above. The first and second polysaccharides can be selected from any of the polysaccharides defined above.

The cationised polysaccharide product according to the invention has a molar ratio of first substituent to second substituent which can be from 10:1 to 1:10, usually from 7:1 to 1:7, suitably from 5:1 to 1:5, preferably from 3:1 to 1:3, and most preferably from 2:1 to 1:2.

The cationised polysaccharide product can have a degree of substitution varying over a wide range; the degree of cationic substitution (DS_{C}) can be from 0.005 to 2.0, suitably from 0.01 to 1.0, and preferably from 0.02 to 0.5; the degree of aromatic substitution (DS_{Ar}) can be from 0.0005 to 2.0, usually from 0.001 to 1.0, suitably from 0.005 to 0.5, and preferably from 0.01 to 0.5; the degree of non-aromatic substitution (DS_{non-Ar}) can be from 0.0005 to 2.0, usually from 0.001 to 1.0, suitably from 0.005 to 0.5, and preferably from 0.01 to 0.5; and the degree of anionic substitution (DS_{A}) can be from 0 to 2.0, suitably from 0 to 1.0, preferably from 0 to 0.5. Usually the charge density of the cationised polysaccharide product is within the range of from 0.01 to 6.0 meq/g of dry polysaccharide, suitably from 0.02 to 5.0 and preferably from 0.05 to 4.0.

The cationised polysaccharide product may consist or essentially consist of one or more polysaccharides according to the invention. The cationised polysaccharide product normally contains a liquid, usually water, and it is usually an aqueous cationised polysaccharide product.

In a preferred embodiment of the invention, the cationised polysaccharide product is in the form of a powder. The powder may contain less than 30% by weight of an aqueous phase, preferably less than 25% by weight, most preferably less than 20% by weight based on the total weight of the polysaccharide product.

In another preferred embodiment of the invention, the cationised polysaccharide product is in the form of an aqueous slurry. The dry content of the polysaccharide in the aqueous slurry can be within the rage of from 10 to 55% by weight, suitable from 20 to 50% by weight, preferably from 25% to 45% by weight, based on the total weight of the polysaccharide product.

In yet another preferred embodiment of the invention, the cationised polysaccharide product is in the form of an aqueous solution. The dry content of the cationised polysaccharide product in aqueous solution can be within the rage of from 10 to 50% by weight by weight, suitably from 15 to 45%, preferably from 20 to 40% by weight, based on the total weight of the dry polysaccharide product.

The present invention also relates to a method for the preparation of a cationised polysaccharide product and a cationised polysaccharide product obtainable by the method. The polysaccharides to be subjected to modification can be non-ionic, anionic, amphoteric or cationic, and the polysaccharides are reacted with aromatic agents and/or non-aromatic agents, which can be non-ionic, cationic or anionic. The polysaccharides can be selected from any of the polysaccharides known in the art including, for example, starches, gums, celluloses, chitins, chitosans, glycans, galactans, glucans, pectins, mannans, dextrins, preferably starches and gums, and mixtures thereof. Examples of suitable starches include potato, corn, wheat, tapioca, rice, waxy maize, etc., preferably potato and corn. Examples of suitable gums are guar gums, tamarind gums, locust bean gums, tara gums, karaya, okra, acacia, xanthan gums etc., preferably guar gums. The non-ionic agents and cationic agents may be reaction products obtained by reaction of halohydrin, epihalohydrin and epichlorohydrin with secondary or tertiary amines. The cationic agents can also comprise quaternary agents. The anionic agents comprise aromatic or non-aromatic agents containing phosphate, phosphonate, sulphate, sulphonate or carboxylic acid groups. The aromatic agent can be reacted with one or more polysaccharides before the polysaccharide is reacted with the non-aromatic agent, simultaneously, in reversed order or separately in case of at least two polysaccharides. In case of separate reactions of at least two polysaccharides, at least one polysaccharide is reacted with an aromatic agent and at least one polysaccharide is reacted with a non-aromatic agent, then the obtained polysaccharides are mixed. The cationised polysaccharide products can be obtained by reaction with the agents in aqueous suspension, pulverulent mixture, aqueous solution or aqueous alcoholic suspension under alkaline conditions. In a preferred embodiment of the present invention the cationised polysaccharide product can be obtained by reaction with one or more cationic agents, e.g. aromatic cationic agents and/or non-aromatic cationic agents.

Aromatic agents of the invention include non-ionic agents, cationic agents, and anionic agents. Examples of suitable agents include:

(I) Non-ionic aromatic agents such as substituted succinic anhydrides having an aromatic group; aralkyl halides, e.g. benzyl chloride and benzyl bromide; ethers, e.g. phenyl glycidyl ether and benzyl glycidyl ether; the reaction products of epichlorohydrin and dialkylamines having at least one substituent comprising an aromatic group.

(II) Cationic aromatic agents such as, the reaction product of epichlorohydrin and tertiary amines having one or more aromatic groups as defined above, including alkaryidialkylamines, e.g. dimethylbenzylamine; arylamines, e.g. pyridine and quinoline. Suitable cationic agents of this type include halohydroxypropyl-N,N-dialkyl-N-alkarylammonium halides and N-glycidyl-N-(alkaryl)-N,N-dialkylammonium chloride, e.g. N-(3-chloro-2-hydroxypropyl)-N-(alkaryl)-N,N-di(lower alkyl)ammonium chloride where the alkaryl and lower alkyl groups are as defined above, particularly N-(3-chloro-2-hydroxypropyl)-N-benzyl-N,N-dimethylammonium chloride; and N-(3-chloro-2-hydroxypropyl) pyridinium chloride. The aromatic cationic agent is preferably 3-chloro-2-hydroxypropyl dimethyl benzyl ammonium chloride or 2,3-epoxipropyl dimethyl benzyl ammonium chloride.

(III) Anionic aromatic agents include agents having an aromatic group attached, such agents can be, for example, phosphate, phosphonate, sulphate, sulphonate or carboxylic acid groups and they are preferably phosphate groups, phosphonate groups or sulphonate groups, e.g. phenyl phosphonic acid and phenyl phosphonic sulphonic acid.

Non-aromatic agents of the invention include non-ionic agents, cationic agents, and anionic agents. Examples of suitable agents include:

(I) Non-ionic non-aromatic agents such as alkylene oxides, e.g. propylene oxide, butylene oxide and iso-butylene oxide; alkylene ethers, e.g. butyl glycidyl ether; alkyl halides, e.g. decyl bromide and docecyl bromide; the reaction products of epichlorohydrin and dialkylamines having at least one substituent comprising a non-aromatic hydrocarbon group as defined above, including 3-dialkylamino-1,2-epoxypropanes.

(II) Cationic non-aromatic agents such as, for example, the reaction product of epichlorohydrin and tertiary amines having non-aromatic hydrocarbon groups as defined above, including trialkylamines. Suitable cationic agents of this type include 2,3-epoxypropyl trialkylammonium halides and halohydroxypropyl trialkylammonium halides, e.g. N-(3-chloro-2-hydroxypropyl)-N-(alkyl)-N,N-di(lalkyl)ammonium chloride and N-glycidyl-N-(alkyl)-N,N-di(alkyl)ammoniumchloride where the non-aromatic hydrocarbon group as defined above, notably octyl, decyl, dodecyl and octadecyl, and the alkyl is methyl, ethyl, propyl or butyl, preferably methyl or ethyl. Preferred non-aromatic hydrocarbon cationic agents include 3-chloro-2-hydroxypropyl trimethyl ammonium chloride or 2,3-epoxipropyl trimethyl ammonium chloride.

(III) Anionic non-aromatic agents such as, for example, agents containing phosphonate groups, e.g. aminochloroethane diethylphosphonic acid; agents containing sulphate groups, e.g. sulfamic acid or SO₃— complexes such as SO₃TMA (trimethylamine), SO₃pyridine; agents containing sulfoalkyl groups, e.g. 2-chloroethane-sulfonates and 3-chloro-2-hydroxypropanesulfonate; agents containing carboxylic alkyl groups, e.g. salts of 1-halocarboxylic acid such as sodium monochloroacetate or sodium chloropropionate; agents containing sulfocarboxyl groups, e.g. 3-chloro-2-sulfopropionic acid; lactones like propionolactone or butyrolactone, acrylonitrile, acid anhydrides such as maleic anhydride, succinic anhydride, phthalic anhydride and the like.

Examples of suitable agents, cationic or amphoteric polysaccharides and cationisation methods include those described in U.S. Pat. Nos. 2,876,217 3,422,087, 4,687,519, 4,785,087, 5,129,989, 5,463,127 and 5,827,372; International Patent Applications WO 94/24169, WO 99/55964, European Patent Application Nos. 0 189 935, 0 303 039, 0 400 361, 0 737 210 and 0 874 000; and S. P. Patel, R. G. Patel and V. S. Patel, Starch/Starke, 41(1989), No. 5, pp. 192-196, the teachings of which are hereby incorporated herein by reference.

The method of the invention comprises reacting one or more polysaccharides with (i) at least one first aromatic agent, and (ii) at least one second non-aromatic agent, at least one of the first and second agents comprising a cationic agent. In a preferred embodiment of the invention, one or more polysaccharides are reacted with at least one first aromatic agent and at least one second non-aromatic agent to form a cationised polysaccharide product. In another preferred embodiment of the invention, one or more first polysaccharides are reacted with at least one first aromatic agent, and one or more second polysaccharides are reacted with at least one second non-aromatic agent, and then the polysaccharides obtained are then mixed to form a cationised polysaccharide product. The first and second polysaccharides can be selected from any of the polysaccharides defined above. In a further preferred embodiment of the invention, one or more polysaccharides are reacted with at least one cationic first aromatic agent, and at least one cationic second non-aromatic agent. The agents are reacted in a molar ratio of aromatic agents to non-aromatic agents that can be within the range of from 10:1 to 1:10, usually from 7:1 to 1:7, suitably from 5:1 to 1:5, preferably from 3:1 to 1:3, and most preferably from 2:1 to 1:2.

The method may also comprise crosslinking of the polysaccharides, e.g. by reaction with epichlorohydrin according to European Patent No. 0 603 727, which renders a higher molecular weight to the polysaccharides and a viscosity increase when the polysaccharides are in solution or slurry. The increase of the viscosity is within the range of from of about 5% to 500%, preferably from about 10% to 400%, and the crosslinking effect provided by the crosslinking agent is within the range of from about 2% to 85%, preferably from about 2% to 60% and more preferably from about 5% to 50% Breakdown Viscosity.

The method may also comprise degradation of the polysaccharides by acid hydrolysis, by the use of peroxides, sodium hypochlorite (NaClO), ozon or enzymes, which renders a lower molecular weight to the polysaccharides and thereby a decrease of the viscosity when the polysaccharides are in solution or slurry. The polysaccharide viscosity can be decreased to viscosities, suitably within the range of from 95% to 0.1%, preferably from 80% to 1% and more preferably of 60% to 5% of the viscosity before degradation.

The method may also comprise both crosslinking and degradation of the polysaccharides, and thereby provide a cationised polysaccharide product in solution or slurry with controlled viscosity.

The present invention further relates to a papermaking process in which one or more cationised polysaccharide products of the invention are added to an aqueous suspension containing cellulosic fibres, or stock, to be dewatered. The cationised polysaccharide products according to the invention can be employed in the papermaking process as drainage and retention aids and as dry strength agents. The term “drainage and retention aid”, as used herein, refers to one or more components (aid, agent or additive) which when being added to an aqueous cellulosic suspension, give better drainage and/or retention than is obtained when not adding said one or more components. The term “dry strength agent”, as used herein, refers to one or more components (aids, agents or additives) which, when being added to a stock, give better dry strength of the paper produced than is obtained when not adding said one or more components.

The process of this invention results in improved drainage and/or retention and hereby the present process makes it possible to increase the speed of the paper machine and to use lower a dosage of additive to give a corresponding drainage and retention effect, thereby leading to an improved papermaking process and economic benefits. Further benefits observed with the present invention include improved dry strength of the paper produced using the cationised polysaccharide product. Hereby it is possible to use a lower dosage of dry strength agent to give a corresponding paper dry strength effect. It is also possible to use high dosages of the cationised polysaccharide product without overcharging the fibre material in order to increase the paper strength and thereby paper quality, since the cationised polysaccharide products according to the invention are very effective also at relatively low cationicity. The process of this invention can be utilised for the treatment of cellulosic suspensions in mills with relatively closed water loops, wherein the white water is repeatedly recycled with the introduction of only low amounts of fresh water. The process is further suitably applied to papermaking processes using cellulosic suspensions having high salt contents, and thus having high conductivity levels, for example processes with extensive white water recycling and limited fresh water supply and/or processes using fresh water having high salt contents.

The cationised polysaccharide product according to the invention can be used in conjunction with additional additives that are beneficial to the overall drainage and/or retention and/or dry strength performance of the process and/or paper produced, thereby forming drainage and retention aids as well as dry strength aids comprising two or more components. Examples of suitable stock additives of this type include anionic materials, e.g. anionic inorganic materials such as, for example, microparticulate materials, e.g. silica-based particles and clays of smectite type, and anionic organic materials such as, for example, anionic organic polymers such as condensation polymers, addition polymers, step-growth polymers, chain-growth polymers, polysaccharides containing anionic groups, synthetic polymers having an aromatic group, naturally occurring aromatic polymers, and modifications thereof. The term “step-growth polymer”, as used herein, refers to a polymer obtained by step-growth polymerisation, also being referred to as step-reaction polymer and step-reaction polymerisation, respectively. Addition polymers are polymers obtained by step-growth addition polymerisation, e.g. anionic polyurethanes which can be prepared from a monomer mixture comprising aromatic isocyanates and/or aromatic alcohols. Condensation polymers i.e. polymers obtained by step-growth condensation polymerisation, e.g. condensates of an aldehyde such as formaldehyde with one or more aromatic compounds, and optional other co-monomers useful in the condensation polymerisation such as urea and melamine. Chain-growth polymers are prepared by polymerisation of one or more monomers having a vinyl group or ethylenically unsaturated bond.

Anionic inorganic materials that can be used according to the invention include anionic silica-based particles and clays of the smectite type. It is preferred that the anionic inorganic particles are in the colloidal range of particle size. Anionic silica-based particles, i.e. particles based on SiO₂ or silicic acid, are preferably used and such particles are usually supplied in the form of aqueous colloidal dispersions, so-called sols. Examples of suitable silica-based particles include different types of polymerised silicic acid, either homopolymerised or co-polymerised. The silica-based particles and/or sols can be modified and contain other elements, e.g. aluminium, nitrogen and/or boron, which can be present in the aqueous phase and/or in the silica-based particles. Suitable silica-based particles of this type include colloidal aluminium-modified silica and aluminium silicates. Mixtures of such suitable silica-based particles can also be used.

Anionic silica-based particles suitably have an average particle size below about 50 nm, preferably below about 20 nm and more preferably in the range of from about 1 to about 10 nm. As conventional in silica chemistry, the particle size refers to the average size of the primary particles, which may be aggregated or non-aggregated. The specific surface area of the silica-based particles suitably is at least 50 m²/g and preferably at least 100 m²/g. Generally, the specific surface area can be up to about 1700 m²/g and preferably up to 1000 m²/g. The specific surface area can be measured by means of titration with NaOH in known manner, e.g. as described by Sears in Analytical Chemistry 28(1956):12, 1981-1983 and in U.S. Pat. No. 5,176,891. The given area thus represents the average specific surface area of the particles.

Suitably the silica-based particles are contained in a sol. The sol may have an S-value in the range of from 5 to 80%, suitably from 5 to 50%, preferably from 8 to 45%, and most preferably from 10 to 30%. Calculation and measuring of the S-value can be performed as described by Iler & Dalton in J. Phys. Chem. 60(1956), 955-957. The S-value indicates the degree of aggregate or microgel formation and a lower S-value is indicative of a higher degree of aggregation.

Suitably the silica-based particles have a molar ratio Si₂O:Na₂O less than 60, usually within the range 5 to 60, and preferably within the range from 8 to 55.

Suitable anionic silica-based particles used as retention and/or drainage aid are disclosed in U.S. Pat. Nos. 4,388,150; 4,927,498; 4,954,220; 4,961,825; 4,980,025; 5,127,994; 5,176,891; 5,368,833; 5,447,604; 5,470,435; 5,543,014; 5,571,494; 5,573,674; 5,584,966; 5,603,805; 5,688,482; and 5,707,493; which are hereby incorporated herein by reference.

Anionic polymers that can be used according to the invention can be selected from step-growth polymers, chain-growth polymers, polysaccharides, naturally occurring aromatic polymers and modifications thereof. The anionic polymers can be linear, branched or cross-linked. Preferably the anionic polymer is water-soluble or water-dispersible. Examples of anionically charged groups that can be present in the anionic polymer as well as in the monomers used for preparing the anionic polymer include groups carrying an anionic charge and acid groups carrying an anionic charge when dissolved or dispersed in water, the groups herein collectively being referred to as anionic groups, such as phosphate, phosphonate, sulphate, sulphonic acid, sulphonate, carboxylic acid, carboxylate, alkoxide and phenolic groups, i.e. hydroxy-substituted phenyls and naphthyls. Groups carrying an anionic charge are usually salts of an alkali metal, alkaline earth or ammonia.

Examples of suitable anionic step-growth polymers include condensation polymers, suitable aromatic compounds containing anionic groups comprises benzene and naphthalene-based compounds containing anionic groups such as phenolic and naphtholic compounds, e.g. phenol, naphthol, resorcinol and derivatives thereof, aromatic acids and salts thereof, e.g. phenylic, phenolic, naphthylic and naphtholic acids and salts, usually sulphonic acids and sulphonates, e.g. benzene sulphonic acid and sulphonate, xylen sulphonic acid and sulphonates, naphthalene sulphonic acid and sulphonate, phenol sulphonic acid and sulphonate.

Examples of further suitable anionic step-growth polymers include addition polymers, e.g. anionic polyurethanes which can be prepared from a monomer mixture comprising aromatic isocyanates and/or aromatic alcohols. Examples of suitable aromatic isocyanates include diisocyanates, e.g. toluene-2,4- and 2,6-diisocyanates and diphenylmethane-4,4′-diisocyanate. Examples of suitable aromatic alcohols include dihydric alcohols, i.e. diols, e.g. bisphenol A, phenyl diethanol amine, glycerol monoterephthalate and trimethylolpropane monoterephthalate. Monohydric aromatic alcohols such as phenol and derivatives thereof may also be employed. The monomer mixture can also contain non-aromatic isocyanates and/or alcohols, usually diisocyanates and diols, for example any of those known to be useful in the preparation of polyurethanes. Examples of suitable monomers containing anionic groups include the monoester reaction products of triols, e.g. trimethylolethane, trimethylolpropane and glycerol, with dicarboxylic acids or anhydrides thereof, e.g. succinic acid and anhydride, terephthalic acid and anhydride, such as glycerol monosuccinate, glycerol monoterephthalate, trimethylolpropane monosuccinate, trimethylolpropane monoterephthalate, N,N-bis-(hydroxyethyl)-glycine, di-(hydroxymethyl)propionic acid, N,N-bis-(hydroxyethyl)-2-aminoethanesulphonic acid, and the like, optionally and usually in combination with reaction with a base, such as alkali metal and alkaline earth hydroxides, e.g. sodium hydroxide, ammonia or an amine, e.g. triethylamine, thereby forming an alkali metal, alkaline earth or ammonium counterion.

Examples of suitable anionic chain-growth polymers include anionic vinyl addition polymers obtained from a mixture of vinylic or ethylenically unsaturated monomers comprising at least one monomer having an aromatic group and at least one monomer having an anionic group, usually co-polymerised with non-ionic monomers such as acrylate- and acrylamide-based monomers. Examples of suitable anionic monomers include (meth)acrylic acid and paravinyl phenol (hydroxy styrene).

Examples of suitable anionic polysaccharides include starches, gums, celluloses, chitins, chitosans, glycans, galactans, glucans, pectins, mannans, dextrins, preferably starches and gums, and mixtures thereof. Examples of suitable starches include potato, corn, wheat, tapioca, rice, waxy maize, etc., preferably potato and corn. Examples of suitable gums are guar gums, tamarind gums, locust bean gums, tara gums, karaya, okra, acacia, xanthan gums etc., preferably guar gums. The anionic groups in the polysaccharide can be native and/or introduced by chemical treatment.

Naturally occurring aromatic anionic polymers and modifications thereof, i.e. modified naturally occurring aromatic anionic polymers, according to the invention include naturally occurring polyphenolic substances that are present in wood and organic extracts of bark of some wood species and chemical modifications thereof, usually sulphonated modifications thereof. The modified polymers can be obtained by chemical processes such as, for example, sulphite pulping and Kraft pulping. Examples of suitable anionic polymers of this type include lignin-based polymers, preferably sulphonated lignins, e.g. ligno-sulphonates, kraft lignin, sulphonated kraft lignin, and tannin extracts.

The average molecular weight of the anionic polymer can vary within wide limits dependent on, inter alia, the type of polymer used, and usually it is at least about 500, suitably above about 2,000 and preferably above about 5,000. The upper limit is not critical; it can be about 200,000,000, usually 150,000,000, suitably 100,000,000 and preferably 10,000,000.

The anionic polymer can have a degree of anionic substitution (DSA) varying over a wide range dependent on, inter alia, the type of polymer used; DSA is usually from 0.01 to 2.0, suitably from 0.02 to 1.8 and preferably from 0.025 to 1.5; and the degree of aromatic substitution (DSAR) can be from 0.001 to 1.0, usually from 0.01 to 0.8, suitably from 0.02 to 0.7 and preferably from 0.025 to 0.5. In case the anionic polymer contains cationic groups, the degree of cationic substitution (DS_{C}) can be, for example, from 0 to 0.2, suitably from 0 to 0.1 and preferably from 0 to 0.05, the anionic polymer having an overall anionic charge. Usually the anionic charge density of the anionic polymer is within the range of from 0.1 to 6.0 meqv/g of dry polymer, suitably from 0.5 to 5.0 and preferably from 1.0 to 4.0.

Examples of suitable anionic aromatic polymers that can be used according to the present invention include those described in U.S. Pat. Nos. 4,070,236 and 5,755,930; and International Patent Application Publication Nos. WO 95/21295, WO 95/21296, WO 99/67310, and WO 00/49227, which are hereby incorporated herein by reference.

The cationised polysaccharide product according to the invention can also be used in conjunction with other additives, e.g. other polysaccharides, aluminium compounds, cationic, non-ionic, and amphoteric synthetic polymers such as, for example, low molecular weight cationic organic polymers, anionic vinyl addition polymers and combinations thereof, including the compounds disclosed in International Patent Application Publication Nos. WO 99/55964, WO 99/55965, and WO 02/12626 which are incorporated herein by reference.

Low molecular weight (hereinafter LMW) cationic organic polymers that can be used according to the invention include those commonly referred to as anionic trash catchers (ATC). The LMW cationic organic polymer can be derived from natural or synthetic sources, and preferably it is an LMW synthetic polymer. Suitable organic polymers of this type include LMW highly charged cationic organic polymers such as polyamines, polyamidoamines, polyethyleneimines, homo- and copolymers based on diallyldimethyl ammonium chloride, (meth)acrylamides and (meth)acrylates. In relation to the molecular weight of the cationised polysaccharide product of this invention, the molecular weight of the LMW cationic organic polymer is preferably lower; it is suitably at least 2,000 and preferably at least 10,000. The upper limit of the molecular weight is usually about 700,000, suitably about 500,000 and preferably about 200,000.

Aluminium compounds that can be used according to the invention include alum, aluminates, aluminium chloride, aluminium nitrate and polyaluminium compounds, such as polyaluminium chlorides, polyaluminium sulphates, polyaluminium compounds containing both chloride and sulphate ions, polyaluminium silicate-sulphates, and mixtures thereof. The polyaluminium compounds may also contain other anions than chloride ions, for example anions from sulphuric acid, phosphoric acid, organic acids such as citric acid and oxalic acid.

The cationised polysaccharide product according to the invention can be added to the suspension as a single polysaccharide having both aromatic and non-aromatic substituents, or as a composition containing different polysaccharides, one of which having at least one first substituent and one of which having at least one second substituent. Alternatively, a polysaccharide having at least one first substituent and a polysaccharide having at least one second substituent are separately added to the suspension.

The cationised polysaccharide product and anionic material according to the invention are preferably separately added to the aqueous suspension containing cellulosic fibres, or stock. Preferably the cationised polysaccharide product and the anionic materials are added to the stock at different positions. The cationised polysaccharide product and the anionic materials can be added in any order. Usually the cationised polysaccharide product is added to the stock prior to adding the anionic material, although the reverse order of addition may also be used. The cationised polysaccharide product can be added to the stock to be dewatered in amounts which can vary within wide limits depending on, inter alia, type of stock, salt content, type of salts, filler content, type of filler, point of addition, etc. Generally the cationised polysaccharide product is added in an amount which give better drainage and/or retention than is obtained when not adding them. The cationised polysaccharide product is usually added in an amount of at least 0.05%, often at least 0.1% by weight, based on dry stock substance, whereas the upper limit is usually 5% and suitably 3% by weight. The anionic material is usually added in an amount of at least 0.001%, often at least 0.005% by weight, based on dry stock substance, whereas the upper limit is usually 3% and suitably 1.5% by weight.

The process of this invention is applicable to all papermaking processes and cellulosic suspensions, and it is particularly useful in the manufacture of paper from a stock that has a high conductivity. In such cases, the conductivity of the stock that is dewatered on the wire is generally at least 0.5 mS/cm, usually at least 1.0 mS/cm, suitably at least 1.5 mS/cm, and preferably at least 2.0 mS/cm. Conductivity can be measured by standard equipment such as, for example, a WTW LF 539 instrument supplied by Christian Berner. The values referred to above are suitably determined by measuring the conductivity of the cellulosic suspension that is fed into or present in the headbox of the paper machine or, alternatively, by measuring the conductivity of white water obtained by dewatering the suspension. High conductivity levels mean high contents of salts (electrolytes) which can be derived from the materials used to form the stock, from various additives introduced into the stock, from the fresh water supplied to the process, etc. Further, the content of salts is usually higher in processes where white water is extensively recirculated, which may lead to considerable accumulation of salts in the water circulating in the process.

The present invention further encompasses papermaking processes where white water is extensively recycled, or recirculated, i.e. with a high degree of white water closure, for example where from 0 to 30 tonnes of fresh water are used per tonne of dry paper produced, usually less than 20, suitably less than 15, preferably less than 10 and notably less than 5 tonnes of fresh water per ton of paper. Recycling of white water obtained in the process suitably comprises mixing the white water with cellulosic fibres and/or optional fillers to form a suspension to be dewatered; preferably it comprises mixing the white water with a suspension containing cellulosic fibres, and optional fillers, before the suspension enters the forming wire for dewatering. The white water can be mixed with the suspension before, between, simultaneous with or after introducing the drainage and retention aids of this invention. Fresh water can be introduced in the process at any stage; for example, it can be mixed with cellulosic fibres in order to form a suspension, and it can be mixed with a thick suspension containing cellulosic fibres to dilute it so as to form a thin suspension to be dewatered, before, simultaneous with or after mixing the suspension with white water.

Further additives which are conventional in papermaking can of course be used in combination with the cationised polysaccharide product according to the invention, such as, for example, other retention and/or drainage aids and other dry strength agents, wet strength agents, optical brightening agents, dyes, sizing agents like rosin-based sizing agents and cellulose-reactive sizing agents, e.g. alkyl and alkenyl ketene dimers, alkyl and alkenyl ketene multimers, and succinic anhydrides, etc. The cellulosic suspension, or stock, can also contain mineral fillers of conventional types such as, for example, kaolin, china clay, titanium dioxide, gypsum, talc and natural and synthetic calcium carbonates such as chalk, ground marble and precipitated calcium carbonate.

The process of this invention is used for the production of paper. The term “paper”, as used herein, of course include not only paper and the production thereof, but also other cellulosic fibre-containing sheet or web-like products, such as for example board and paperboard, and the production thereof. The process can be used in the production of paper from different types of suspensions of cellulose-containing fibres and the suspensions should suitably contain at least 25% by weight and preferably at least 50% by weight of such fibres, based on dry substance. The suspension can be based on fibres from chemical pulp such as sulphate, sulphite and organosolv pulps, mechanical pulp such as thermomechanical pulp, chemo-thermomechanical pulp, refiner pulp and groundwood pulp, from both hardwood and softwood, and can also be based on recycled fibres, optionally from de-inked pulps, and mixtures thereof.

The invention also relates to uses of the polysaccharide product in papermaking processes. In a preferred embodiment of this invention, the cationised polysaccharide product is capable of functioning as a dry strength agent. In another preferred embodiment of this invention, the polysaccharide product is capable of functioning as drainage and/or retention aid. In a third preferred embodiment the polysaccharide product is capable of functioning as both dry strength agent and drainage and/or retention aid.

The invention is further illustrated in the following Examples which, however, are not intended to limit the same. Parts and % relate to parts by weight and % by weight, respectively, unless otherwise stated.

### EXAMPLE 1

Cationic polysaccharide products used in the tests were prepared by reacting native potato starch with one or more quaternising agents according to the general procedure described in EP-A 0 189 935 and WO 99/55964. The cationic starches used in the tests, hereinafter also collectively referred to as C1, C2, C3 and C4 according to the invention and ATC1, Ref. 1, Ref. 2, Ref. 3, Ref. 4, Ref. 5, Ref. 6 and Ref. 7 intended for comparison purposes, were the following:

C1: Cationic starch obtained by quarternisation of native potato starch with 3-chloro-2-hydroxypropyl dimethyl benzyl ammonium chloride to DS_{Ar} 0.025 and with 2,3-epoxypropyl trimethyl ammonium chloride to DS_{non-Ar} 0.025, and DS_{C} was 0.05.

C2: Cationic starch obtained by quarternisation of native potato starch with 3-chloro-2-hydroxypropyl dimethyl benzyl ammonium chloride to DS_{Ar} 0.032 and with 2,3-epoxypropyl trimethyl ammonium chloride to DS_{non-Ar} 0.008, and DS_{C} was 0.04.

C3: Cationic starch mixture containing 1 part starch obtained by quarternisation of native potato starch with 3-chloro-2-hydroxypropyl dimethyl benzyl ammonium chloride to DS_{C} 0.05 mixed with 1 part starch obtained by quarternisation of native potato starch with 2,3-epoxypropyl trimethyl ammonium chloride to DS_{C} 0.05.

C4: Cationic starch obtained by quarternisation of native potato starch with 3-chloro-2-hydroxypropyl dimethyl benzyl ammonium chloride to DS_{Ar} 0.065 and also modified with 2,3-epoxy-2-methyl-propane to DS_{non-Ar} 0.01, and DS_{C} was 0.065.

C5: Cationic starch obtained by quarternisation of native potato amylopectin starch with 3-chloro-2-hydroxypropyl dimethyl benzyl ammonium chloride to DS_{Ar} 0.07 and with 2,3-epoxypropyl trimethyl ammonium chloride to DS_{non-Ar} 0.59, and DS_{C} was 0.66.

C6: Cationic starch obtained by quarternisation of native potato amylopectin starch with 3-chloro-2-hydroxypropyl dimethyl benzyl ammonium chloride to DS_{Ar} 0.14 and with 2,3-epoxypropyl trimethyl ammonium chloride to DS_{non-Ar} 0.54, and DS_{C} was 0.68.

ATC1: Cationic polyamine having a molecular weight of about 50,000.

Ref. 1: Cationic starch obtained by quarternisation of native potato starch with 2,3-epoxypropyl trimethyl ammonium chloride to DS_{C} 0.08.

Ref. 2: Cationic starch obtained by quarternisation of native potato starch with 2,3-epoxypropyl trimethyl ammonium chloride to DS_{C} 0.18.

Ref. 3: Cationic starch obtained by quarternisation of native potato starch with 3-chloro-2-hydroxypropyl dimethyl benzyl ammonium chloride to DS_{C} 0.09.

Ref. 4: Cationic starch obtained by quarternisation of native potato starch with 2,3-epoxypropyl trimethyl ammonium chloride to DS_{C} 0.05.

Ref. 5: Cationic starch obtained by quarternisation of native potato starch with 3-chloro-2-hydroxypropyl dimethyl benzyl ammonium chloride to DS_{C} 0.05.

Ref 6: Cationic starch obtained by quarternisation of native potato starch with 2,3-epoxypropyl trimethyl ammonium chloride to DS_{C} 0.04.

Ref. 7: Cationic starch obtained by quarternisation of native potato starch with 2,3-epoxypropyl trimethyl ammonium chloride to DS_{non-Ar} 0.065 and also modified with 2,3-epoxy-2-methyl-propane to DS_{non-Ar} 0.01, DS_{tot(non-Ar)} 0.075, and DS_{C} was 0.065.

Ref. 8: Cationic starch obtained by quarternisation of native potato amylopectin starch with 2,3-epoxypropyl trimethyl ammonium chloride to DS_{C} 0.65.

Ref. 9: Cationic starch obtained by quarternisation of native potato amylopectin starch with 3-chloro-2-hydroxypropyl dimethyl benzyl ammonium chloride to DS_{C} 0.65. C1, C2, C3, C4, C5, ATC1, Ref. 1, Ref. 2, Ref. 3, Ref. 4, Ref. 5 and Ref. 6 were all used as dilute aqueous solutions in all tests.

Anionic components used in the tests were anionic silica sol (A1) and anionic polycondensate (A2). The anionic components used in the tests were the following:

A1: Silica sol of the type described in U.S. Pat. No. 5,368,833, having an S-value of 25% and containing silica particles with a specific surface area of 900 m²/g which are surface-modified with aluminium to a degree of 5%.

A2: Anionic polycondensate of formaldehyde with naphthalene sulphonate, molecular weight about 20,000.

A1 and A2 were used as sols or dilute aqueous solutions.

### EXAMPLE 2

Dry strength performance was evaluated with a Dynamic Sheet Former (Formette Dynamique), supplied by Fibertech AB, Sweden, and a Burst Strength Tester supplied by Lorentzen & Wettre, Sweden.

The furnish used in the tests was based on 100% by weight of recycled waste mill furnish. The furnish consistency was 0.5% and the conductivity was adjusted by addition of calcium chloride to 2.7 mS/cm and further by sodium chloride to 4.0 mS/cm.

The furnish was stirred with a high shear stirrer at a speed of 700 rpm and the starches were added after 0.5 min followed by 5 min of stirring in the mixing chest.

Paper sheets were formed in the Dynamic Sheet Former by pumping the furnish from the mixing chest through a traversing nozzle into the rotating drum onto the water film on top of the wire, draining the stock to form a sheet, pressing and drying the sheet. The sheets were evaluated in the Burst Strength Tester. The burst strength index increase values were calculated and compared.

Table 1 shows Burst Strength Index Increase of the sheets obtained at various starch dosages, calculated as dry starch on dry stock system.

TABLE 1

| | | Burst Strength Index Increase | | | |
|---|---|---|---|---|---|
| Test | Starch Dosage | [%] | | | |
| No. | [kg/t] | C1 | Ref. 1 | Ref. 2 | Ref. 3 |
| 1 | 5.0 | 17.1 | 8.8 | 10.7 | 8.6 |
| 2 | 10.0 | 20.1 | 15.7 | 3.5 | 9.5 |
| 3 | 20.0 | 30.4 | 17.8 | 18.8 | 17.3 |
| 4 | 30.0 | 29.7 | 15.9 | 13.5 | 20.0 |

### EXAMPLE 3

Dry strength performance was evaluated using the equipment according to Example 2. The furnish used in the tests was based on 100% by weight of recycled waste mill stock. The thick stock consistency was 3.6% and the conductivity of the thick stock was adjusted by addition of calcium chloride to 3.0 mS/cm. The white water consistency was 0.1% and the conductivity of the white water was adjusted by addition of calcium chloride to 4.0 mS/cm.

The thick stock was stirred with a high shear stirrer at a speed of 700 rpm and the starches were added after 0.5 min. After 6.5 min the thick stock was mixed with the white water in the mixing chest for 2 min, creating the furnish.

Paper sheets were formed in the Dynamic Sheet Former by pumping the furnish from the mixing chest through a traversing nozzle into the rotating drum onto the water film on top of the wire, draining the stock to form a sheet, pressing and drying the sheet. The sheets were evaluated in the Burst Strength Tester. The burst strength index values were calculated and compared.

Table 2 shows Burst Strength Index of the sheets obtained at various starch dosages, calculated as dry starch on dry stock system.

TABLE 2

| | | |
|---|---|---|
| | | Burst Strength Index |
| Test | Starch Dosage | [MN/kg] |

| No. | [kg/t] | C1 | Ref. 4 |
|---|---|---|---|
| 1 | 0 | 2.37 | 2.37 |
| 2 | 5.0 | 2.77 | 2.49 |
| 3 | 10.0 | 2.86 | 2.47 |
| 4 | 20.0 | 2.92 | 2.67 |

### EXAMPLE 4

Drainage performance was evaluated by means of a Dynamic Drainage Analyser (DDA), available from Akribi, Sweden, which measures the time for draining a set volume of stock through a wire when removing a plug and applying vacuum to that side of the wire opposite to the side on which the stock is present.

Retention performance was evaluated by means of a nephelometer by measuring the turbidity of the filtrate, the white water, obtained by draining the stock. The turbidity was measure in NTU (Nephelometric Turbidity Units).

The furnish used in the tests was based on 56% by weight of peroxide bleached TMP/SGW pulp (80/20), 14% by weight of bleached birch/pine sulphate pulp (60/40) refined to 2000 CSF and 30% by weight of china clay. To the stock was added a colloidal fraction, bleach water from an SC mill. Stock consistency was 0.12%. Conductivity of the stock was adjusted by addition of calcium chloride to 1.0 mS/cm.

The stock was stirred in a baffled jar at a speed of 1500 rpm throughout the tests and chemicals additions were conducted as follows: i) adding cationic starch to the stock following by stirring for 30 seconds, ii) adding anionic component to the stock followed by stirring for 15 seconds, iii) draining the stock while automatically recording the drainage time.

Table 3 shows the dewatering effect at various dosages of cationised starch, calculated as dry starch on dry stock system, and silica-based particles A1, calculated as SiO₂ and based on dry stock system.

TABLE 3

| | Starch | A1 | Dewatering | | Turbidity | |
|---|---|---|---|---|---|---|
| Test | Dosage | Dosage | Times [sec] | | [NTU] | |
| No. | [kg/t] | [kg/t] | C1 | Ref. 1 | C1 | Ref. 1 |
| 1 | 0 | 0 | 19.4 | 19.4 | 90 | 90 |
| 2 | 5 | 3 | 16.5 | 18.1 | 57 | 59 |
| 3 | 10 | 3 | 14.5 | 15.8 | 47 | 52 |
| 4 | 15 | 3 | 11.4 | 14.5 | 43 | 45 |
| 5 | 20 | 3 | 12.0 | 13.2 | 40 | 45 |

### EXAMPLE 5

Fines retention was measured in the Britt Dynamic Drainage Jar, BDDJ, available from e.g. Paper Materials Inc., U.S., which measures the first pass retention of fines for a set volume of stock on a wire.

The furnish used in the tests was based on 56% by weight of peroxide bleached TMP/SGW pulp (80/20), 14% by weight of bleached birch/pine sulphate pulp (60/40) refined to 2000 CSF and 30% by weight of china clay. To the stock was added a colloidal fraction, bleach water from an SC mill. Stock consistency was 0.5%. The conductivity of the stock was adjusted by addition of calcium chloride to 3.0 mS/cm.

The stock was stirred in a baffled jar at a speed of 1000 rpm throughout the tests and chemicals additions were conducted as follows: i) adding polysaccharide to the stock following by stirring for 30 seconds, ii) adding anionic inorganic particles to the stock followed by stirring for 15 seconds, iii) draining the stock during 30 seconds, recording the volume and measuring the dry content of that volume.

Table 4 shows the fines retention effect at various dosages of cationised starch, calculated as dry starch on dry stock system, and silica-based particles, calculated as SiO₂ and based on dry stock system.

TABLE 4

| | Starch | A1 | Fines retention | | |
|---|---|---|---|---|---|
| Test | Dosage | Dosage | [%] | | |
| No. | [kg/t] | [kg/t] | C1 | Ref 4 | Ref 5 |
| 1 | 0 | 0 | 13.7 | 13.7 | 13.7 |
| 2 | 5 | 3 | 41.6 | 29.3 | 27.0 |
| 3 | 10 | 3 | 47.2 | 40.6 | 37.6 |
| 4 | 15 | 3 | 52.7 | 45.9 | 43.3 |

### EXAMPLE 6

Drainage and retention performance was evaluated in a manner similar to Example 4. The furnish used in the tests was based on 70% by weight of bleached birch/pine sulphate pulp (60/40) refined to 2000 CSF and 30% by weight of calcium carbonate. To the stock was added a colloidal fraction, bleach water from an SC mill. Stock consistency was 0.28%. Conductivity of the stock was adjusted by addition of sodium sulphate to 0.45 mS/cm and further by calcium chloride to 2.2 mS/cm.

Table 5 shows the dewatering effect at a constant dosage of cationised starch, calculated as dry starch on dry stock system, and various dosages of the anionic component, A2, based on dry stock system.

TABLE 5

| | Starch | A2 | Dewatering | | Turbidity | |
|---|---|---|---|---|---|---|
| Test | Dosage | Dosage | Times [sec] | | [NTU] | |
| No. | [kg/t] | [kg/t] | C2 | Ref. 6 | C2 | Ref. 6 |
| 1 | 15 | 0 | 18.1 | 18.1 | — | — |
| 2 | 15 | 1 | 12.5 | 17.6 | 93 | 118 |
| 3 | 15 | 2 | 11.8 | 16.1 | 87 | 109 |

### EXAMPLE 7

Drainage and retention performance was evaluated in a manner similar to Example 4. The furnish used in the tests was based on 100% by weight of unbleached softwood Kraft pulp. Stock consistency was 0.43%. Conductivity of the stock was adjusted by addition of calcium chloride to 5.4 mS/cm.

Table 6 shows the dewatering effect at various dosages of cationised starch, calculated as dry starch on dry stock system, and silica-based particles, calculated as SiO₂ and based on dry stock system.

TABLE 6

| | Starch | A1 | Dewatering Times | | |
|---|---|---|---|---|---|
| Test | Dosage | Dosage | [sec] | | |
| No. | [kg/t] | [kg/t] | C3 | Ref 4 | Ref 5 |
| 1 | 0 | 2 | 15.4 | 15.4 | 15.4 |
| 2 | 2.5 | 2 | 14.1 | 14.8 | 14.3 |
| 3 | 5 | 2 | 11.6 | 13.1 | 12.8 |

### EXAMPLE 8

Drainage and retention performance was evaluated in a manner similar to Example 4. The furnish used in the tests was based on 56% by weight of peroxide bleached TMP/SGW pulp (80/20), 14% by weight of bleached birch/pine sulphate pulp (60/40) refined to 200° CSF and 30% by weight of china clay. To the stock was added a colloidal fraction, bleach water from an SC mill. Stock consistency was 0.12%. Conductivity of the stock was adjusted by addition of calcium chloride to 3.5 mS/cm.

Table 7 shows the dewatering effect at various dosages of cationised starch, calculated as dry starch on dry stock system, and silica-based particles, calculated as SiO₂ and based on dry stock system.

TABLE 7

| | Starch | A1 | Dewatering | | Turbidity | |
|---|---|---|---|---|---|---|
| Test | Dosage | Dosage | Times [sec] | | [NTU] | |
| No. | [kg/t] | [kg/t] | C4 | Ref. 7 | C4 | Ref. 7 |
| 1 | 0 | 0 | 20.8 | 20.8 | 94 | 94 |
| 2 | 5 | 3 | 17.7 | 17.9 | 63 | 64 |
| 3 | 10 | 3 | 14.7 | 17.3 | 61 | 61 |
| 4 | 15 | 3 | 14.9 | 19.2 | 57 | 64 |

### EXAMPLE 9

Drainage performance was evaluated in a manner similar to Example 4. The furnish used in the tests was based on 56% by weight of peroxide bleached TMP/SGW pulp (80/20), 14% by weight of bleached birch/pine sulphate pulp (60/40) refined to 2000 CSF and 30% by weight of china clay. To the stock was added a colloidal fraction, bleach water from an SC mill. Stock consistency was 0.16%. Conductivity of the stock was adjusted by addition of calcium chloride to 5.0 mS/cm.

Retention performance was evaluated by means of a Hach 2100P by measuring the turbidity of the filtrate, the white water, obtained by draining the stock. The turbidity was measure in NTU (Nephelometric Turbidity Units).

Table 8 shows the dewatering effect at various dosages of cationised starch, calculated as dry starch on dry stock system.

TABLE 8

| | Starch Dosage | Dewatering Times [sec] | | | |
|---|---|---|---|---|---|
| Test No. | [kg/t] | C5 | C6 | Ref. 8 | Ref. 9 |
| 1 | 0 | 27.6 | 27.6 | 27.6 | 27.6 |
| 2 | 3 | 15.9 | 15.5 | 16.9 | 22.1 |
| 3 | 5 | 13.1 | 12.4 | 13.5 | 17.5 |

Table 9 shows the retention effect at various dosages of cationised starch, calculated as dry starch on dry stock system.

TABLE 9

| | Starch Dosage | Turbidity [NTU] | | | |
|---|---|---|---|---|---|
| Test No. | [kg/t] | C5 | C6 | Ref. 8 | Ref. 9 |
| 1 | 0 | 188 | 188 | 188 | 188 |
| 2 | 3 | 121 | 131 | 131 | 161 |
| 3 | 5 | 119 | 118 | 130 | 148 |

1. A cationised polysaccharide product comprising a polysaccharide having at least one first substituent having an aromatic group and at least one second substituent having no aromatic group, wherein the first substituent and second substituent are present in a molar ratio within the range of from 10:1 to 1:10.

2. The cationised polysaccharide product of claim 1, wherein the first substituent and second substituent are present in a molar ratio within the range of from 7:1 to 1:7.

3. The cationised polysaccharide product of claim 1, wherein it has a cationic charge density within the range of from 0.05 to 4.0 meq/g.

4. The cationised polysaccharide product of claim 1, wherein the first substituent comprises the following general structural formula (I): 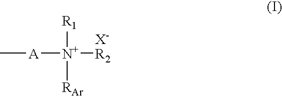
wherein A is a group attaching N to the polysaccharide, R₁ and R₂ are individually H or alkyl having from 1 to 3 carbon atoms, R_{Ar} is an aromatic group containing 1 to 12 carbon atoms, or, alternatively, R₁, R₂, and R_{Ar}, together with N form an aromatic group, and X⁻ is a counterion.

5. The cationised polysaccharide product of claim 1, wherein the first substituent comprises —CH₂—CH(OH)—CH₂—N⁺((CH₃)₂)CH₂C₆H₅Cl⁻.

6. The cationised polysaccharide product of claim 1, wherein the first substituent comprises a benzyl group.

7. The cationised polysaccharide product of claim 1, wherein the second substituent comprises the general structural formula (II): 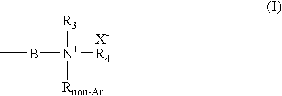
wherein B is a group attaching N to the polysaccharide, R₃ and R₄ are individually H or alkyl having from 1 to 3 carbon atoms; R_{non-Ar} is a non-aromatic group containing 1 to 4 carbon atoms; and X⁻ is a counterion.

8. The cationised polysaccharide product of claim 1, wherein the second substituent comprises —CH₂—CH(OH)—CH₂—N⁺((CH₃)₃)Cl⁻.

9. The cationised polysaccharide product of claim 1, wherein the first substituent comprises —CH₂—CH(OH)—CH₂—N⁺((CH₃)₂)CH₂C₆H₅Cl⁻ and the second substituent comprises —CH₂—CH(OH)—CH₂—N⁺((CH₃)₃)Cl⁻.

10. The cationised polysaccharide product of claim 9, wherein the first substituent and second substituent are present in a molar ratio within the range of from 7:1 to 1:7.

11. A cationised polysaccharide product comprising one or more polysaccharides having at least one first substituent having an aromatic group and one or more polysaccharides having at least one second substituent having no aromatic group.

12. The cationised polysaccharide product of claim 11, wherein it has an overall cationic charge density within the range of from 0.05 to 4.0 meq/g.

13. The cationised polysaccharide product of claim 11, wherein the first substituent has the following general structural formula (I): 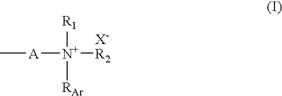
wherein A is a group attaching N to the polysaccharide, R₁ and R₂ are individually H or alkyl having from 1 to 3 carbon atoms, R_{Ar} is an aromatic group containing 1 to 12 carbon atoms, or, alternatively, R₁, R₂, and R_{Ar} together with N form an aromatic group, and X⁻ is a counterion.

14. The cationised polysaccharide product of claim 11, wherein the first substituent comprises —CH₂—CH(OH)—CH₂—N⁺((CH₃)₂)CH₂C₆H₅Cl⁻.

15. The cationised polysaccharide product of claim 11, wherein the first substituent comprises a benzyl group.

16. The cationised polysaccharide product of claim 11, wherein the second substituent has the general structural formula (II): 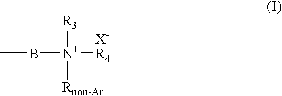
wherein B is a group attaching N to the polysaccharide, R₃ and R₄ are individually H or alkyl having from 1 to 3 carbon atoms; R_{non-Ar} is a non-aromatic group containing 1 to 4 carbon atoms; and X⁻ is a counterion.

17. The cationised polysaccharide product of claim 11, wherein the second substituent comprises —CH₂—CH(OH)—CH₂—N⁺((CH₃)₃)Cl⁻.

18. The cationised polysaccharide product of claim 11, wherein the first substituent comprises —CH₂—CH(OH)—CH₂—N⁺((CH₃)₂)CH₂C₆H₅Cl⁻ and the second substituent comprises —CH₂—CH(OH)—CH₂—N⁺((CH₃)₃)Cl⁻.

19. A cationised polysaccharide product comprising a polysaccharide having a degree of aromatic substitution (DS_{Ar}) within the range of from 0.0005 to 2.0 and a degree of non-aromatic substitution (DS_{non-Ar}) within the range of from 0.0005 to 2.0.

20. The cationised polysaccharide product of claim 19, wherein the polysaccharide has a degree of cationic substitution (DS_{C}) within the range of from 0.02 to 0.5.

21. A cationised polysaccharide product obtained by reacting one or more polysaccharides with:
(i) at least one first aromatic agent; and
(ii) at least one second non-aromatic agent;
wherein the first aromatic agent and second non-aromatic agent are reacted in a molar ratio within the range of from 10:1 to 1:10.

22. The cationised polysaccharide product of claim 21, wherein the first aromatic agent and second non-aromatic agent are reacted in a molar ratio within the range of from 7:1 to 1:7.

23. The cationised polysaccharide product of claim 21, wherein the first aromatic agent is a cationic reaction product of epichlorohydrin and a tertiary amine having one or more aromatic groups.

24. The cationised polysaccharide product of claim 21, wherein second non-aromatic agents is a cationic reaction product of epichlorohydrin and a tertiary amine having non-aromatic hydrocarbon groups.

25. A cationised polysaccharide product obtained by reacting:
(i) a first polysaccharide with at least one first aromatic agent; and
(ii) a second polysaccharide with at least one second non-aromatic agent;
and then mixing the polysaccharides obtained.

26. The cationised polysaccharide product of claim 25, wherein the first aromatic agent and second non-aromatic agent are reacted in a molar ratio within the range of from 7:1 to 1:7.

27. The cationised polysaccharide product of claim 25, wherein the first aromatic agent is a cationic reaction product of epichlorohydrin and a tertiary amine having one or more aromatic groups.

28. The cationised polysaccharide product of claim 25, wherein second non-aromatic agents is a cationic reaction product of epichlorohydrin and a tertiary amine having non-aromatic hydrocarbon groups.
